(19) United States
(12) Patent Application Publication
(10) Pub. No.: US 2014/0343147 A1
(43) Pub. Date: Nov. 20, 2014

(54) NUTRITIONAL SUPPLEMENT FOR THE ENHANCEMENT OF MUSCLE PERFORMANCE AND RECOVERY AND METHODS OF USE THEREOF

(71) Applicant: Maximum Human Performance, LLC, West Caldwell, NJ (US)

(72) Inventors: Gerard Dente, III, Cedar Grove, NJ (US); Robert Thoburn, Delray Beach, FL (US)

(21) Appl. No.: 14/450,571

(22) Filed: Aug. 4, 2014

Related U.S. Application Data
(62) Division of application No. 13/548,626, filed on Jul. 13, 2012.
(60) Provisional application No. 61/507,702, filed on Jul. 14, 2011.

(51) Int. Cl. A61K 31/198; A61K 31/19

(52) U.S. Cl. CPC A61K 31/198; A61K 31/19
USPC 514/561

(57) ABSTRACT

Described herein are nutritional supplements containing α-hydroxy-isocaproic acid and aspartic acid (e.g., DAA) and their methods of use in enhancing muscle performance and/or recovery after exercise. The nutritional supplements can be used to enhance muscle building and lean body mass, enhance muscle and physical performance, enhance recovery, and prevent catabolism (muscle atrophy or wasting) when administered to a mammalian subject.

### CROSS-REFERENCE TO RELATED APPLICATIONS

This application is a divisional of U.S. application Ser. No. 13/548,626, filed on Jul. 13, 2012, which claims priority to U.S. Provisional Application No. 61/507,702 filed on Jul. 14, 2011, which is incorporated herein by reference in its entirety.

### BACKGROUND

Nutritional supplements are used to increase skeletal muscle mass and/or strength, both in exercising and non-exercising individuals. For example, muscle mass and strength are lost during the aging process, and mitigating such losses is an important part of a healthy aging process. Supplements can help build muscle mass and strength that is lost over time and/or mitigate future losses.

In the fields of sports and physical exercise, an increase in lean body mass, a tissue compartment that includes skeletal muscle, without an accompanying increase in fat mass is desired. For this purpose, and to enhance recovery from exercise, a variety of nutritional supplements are commercially available. Immediately after strenuous exercise both muscle protein synthesis and breakdown may be increased, whereas protein balance (protein synthesis—protein breakdown) may be negative due to the predominance of protein breakdown. The response of muscle protein metabolism to a resistance exercise bout, for example, may last 24-48 hours.

Another effect of exercise is to induce muscle soreness, also known as delayed-onset muscle soreness (DOMS). DOMS is the sensation of muscular discomfort and pain during active contractions, which occurs in a delayed fashion after strenuous exercise. The soreness and accompanying muscle damage are more pronounced when the exercise performed is new to the individual.

Nutritional supplements are typically designed to compensate for reduced levels of nutrients in the diet. In particular, in the field of sports and physical exercise, nutritional supplements that improve physical performance and recovery from exercise are increasingly important. The most common supplements currently used are mixtures containing carbohydrate, creatine, protein, dietarily essential and non-essential amino acids, vitamins and/or minerals. Despite advancements in the science of nutrition, simpler and more effective means of enhancing muscle mass and strength, physical performance and recovery from exercise with minimal undesirable, or, “side” effects are needed.

Disclosed herein are compositions and methods for the enhancement of muscle mass and strength, physical performance, and recovery from exercise.

### SUMMARY

In one aspect, a nutritional supplement comprises a-hydroxy-isocaproic acid, a physiologically acceptable ester or amide derivative of a-hydroxy-isocaproic acid, or a salt of any one of the foregoing; aspartic acid, a physiologically acceptable ester or amide derivative of aspartic acid, or a salt of any one of the foregoing; and optionally a pharmaceutically acceptable excipient.

In another aspect, a method for enhancing muscle mass, muscle strength, physical performance, recovery from exercise, or a combination thereof in a mammal comprises administering to the mammal, before exercise, during exercise, after exercise, or a combination thereof, a nutritional supplement comprising: α-hydroxy-isocaproic acid, a physiologically acceptable ester or amide derivative of α-hydroxy-isocaproic acid, or a salt of any one of the foregoing; aspartic acid, a physiologically acceptable ester or amide derivative of aspartic acid, or a salt of any one of the foregoing; and optionally a pharmaceutically acceptable excipient.

In yet another aspect, a method of enhancing or preserving muscle mass, muscle strength, physical performance, or a combination thereof in a mammal in need thereof comprises administering to the mammal a nutritional supplement comprising: α-hydroxy-isocaproic acid , a physiologically acceptable ester or amide derivative of α-hydroxy-isocaproic acid, or a salt of any one of the foregoing; aspartic acid, a physiologically acceptable ester or amide derivative of aspartic acid, or a salt of any one of the foregoing; and optionally a pharmaceutically acceptable excipient

### DETAILED DESCRIPTION

Disclosed herein are nutritional supplements comprising a novel combination of active agents and methods of using the compositions to enhance muscle mass, muscle strength, physical performance, recovery from exercise, to prevent loss of muscle mass and strength, or a combination thereof. The compositions are particularly suitable for use by athletes and in the treatment or prevention of the loss of muscle mass and strength associated with normal aging (sarcopenia), reductions in testosterone associated with normal aging (andropause), and exercise-induced muscle soreness such as delayed-onset muscle soreness (DOMS).

The nutritional supplements described herein include α-hydroxy-isocaproic acid, a physiologically acceptable ester or amide derivative, or a salt of any one of the foregoing; aspartic acid, a physiologically acceptable ester or amide derivative, or a salt of any one of the foregoing; and optionally a pharmaceutically acceptable excipient. As used herein, unless noted otherwise, the term α-hydroxy-isocaproic acid (HICA) includes its D- and L- isomers and a racemic mixture thereof (DL-α-hydroxy-isocaproic acid). Similarly, the term aspartic acid includes its D- and L-isomers and a racemic mixture thereof (DL-α-hydroxy-isocaproic acid).

It was unexpectedly found by the inventors herein that by administering a combination of an agent that increases protein synthesis, or anabolism, and/or reduces protein breakdown, or, catabolism (HICA) with an agent that increases levels of the anabolic and androgenic hormone testosterone (aspartic acid, or, DAA for D-aspartic acid), an unexpectedly synergistic improvement in muscle mass and strength, physical performance, and recovery from exercise is observed compared to the administration of either agent alone. Without being held to theory, it is believed that the dietarily essential amino acid leucine, which HICA is a metabolite of, activates the mammalian target of rapamycin (mTOR) and downstream targets such as p70s6 kinase. mTOR is a complex that regulates input from pathways involving insulin, growth factors and amino acids. When leucine concentrations increase, mTOR and downstream targets such as p70s6 kinase are activated and the rate of skeletal muscle protein synthesis increases. mTOR and p70s6 kinase are thought to play important roles in mediating exercise-induced increases in muscle size, or hypertrophy. Aspartic acid, specifically DAA, stimulates the release of testosterone. Testosterone, in turn, acts permissively on muscle tissue growth by increasing protein synthesis and/or reducing protein catabolism. The enhancement of muscle growth via the effects of both HICA and aspartic acid results in a synergistic improvement in muscle mass and strength, physical performance, and recovery from exercise, for example.

HICA (α-hydroxy-isocaproic acid; synonyms: 2-hydroxy-4-methylvaleric acid, leucic acid) is a normally occurring metabolite in mammalian organisms including humans. It is a metabolite of the branched-chain amino acid leucine, which is considered essential in the human diet. MCA is non-toxic with an LD₅₀ (iv. in mice, Na-salt) of 650 mg/kg. It is commercially available as colorless crystals that possess a sweet and sour taste and are soluble in water and alcohol. HICA enhances performance and/or recovery of the muscles in a state of stress induced by physical exercise, such as long-term strenuous physical exercise, and in states involving the loss or atrophy of muscle such as following surgery, ruptures, or other injuries or disorders which are associated with decreased muscle anabolism and/or increased muscle catabolism.

Exemplary salts of HICA and its derivatives include physiologically acceptable inorganic salts, such as ammonium, sodium, potassium, calcium, magnesium and similar salts, and physiologically acceptable organic salts. In one embodiment, a HICA salt is the pyridoxine salt.

D-aspartic acid (DAA) is an endogenous amino acid found in endocrine and nervous tissues in humans, and other animals, including the pituitary and testes. A daily dose of D-aspartic acid in humans and rats was shown to increase luteinizing hormone and testosterone levels. Testosterone is believed to create an anabolic atmosphere within the body that encourages muscle growth, heightens muscle performance and improves body composition.

Exemplary salts of aspartic acid and its derivative include the calcium and sodium salts. DAA also includes the free acid, D-aspartic acid.

In one embodiment, a nutritional supplement comprises α-hydroxy-isocaproic acid, a physiologically acceptable ester or amide derivative of α-hydroxy-isocaproic acid, or a salt of any one of the foregoing; aspartic acid, a physiologically acceptable ester or amide derivative of aspartic acid, or a salt of any one of the foregoing; and optionally a pharmaceutically acceptable excipient. In another embodiment, a nutritional supplement consists essentially of α-hydroxy-isocaproic acid, a physiologically acceptable ester or amide derivative of α-hydroxy-isocaproic acid, or a salt of any one of the foregoing; aspartic acid, a physiologically acceptable ester or amide derivative of aspartic acid, or a salt of any one of the foregoing; and optionally a pharmaceutically acceptable excipient. In yet another embodiment, a nutritional supplement consists of α-hydroxy-isocaproic acid, a physiologically acceptable ester or amide derivative of α-hydroxy-isocaproic acid, or a salt of any one of the foregoing; aspartic acid, a physiologically acceptable ester or amide derivative of aspartic acid, or a salt of any one of the foregoing; and optionally a pharmaceutically acceptable excipient.

In one embodiment, a nutritional supplement comprises a muscle mass, strength, physical performance, or recovery enhancing amount of α-hydroxy-isocaproic acid, a physiologically acceptable ester or amide derivative of α-hydroxy-isocaproic acid, or a salt of any one of the foregoing; a muscle mass, strength, physical performance, or recovery enhancing amount of aspartic acid, a physiologically acceptable ester or amide derivative of aspartic acid, or a salt of any one of the foregoing; and optionally a pharmaceutically acceptable excipient. In another embodiment, a nutritional supplement consists essentially of a muscle mass, strength, physical performance, or recovery enhancing amount of α-hydroxy-isocaproic acid, a physiologically acceptable ester or amide derivative of α-hydroxy-isocaproic acid, or a salt of any one of the foregoing; a muscle mass, strength, physical performance, or recovery enhancing amount of aspartic acid, a physiologically acceptable ester or amide derivative of aspartic acid, or a salt of any one of the foregoing; and optionally a pharmaceutically acceptable excipient. In yet another embodiment, a nutritional supplement consists of a muscle mass, strength, physical performance, or recovery enhancing amount of α-hydroxy-isocaproic acid, a physiologically acceptable ester or amide derivative of α-hydroxy-isocaproic acid, or a salt of any one of the foregoing; a muscle mass, strength, physical performance, or recovery enhancing amount of aspartic acid, a physiologically acceptable ester or amide derivative of aspartic acid, or a salt of any one of the foregoing; and optionally a pharmaceutically acceptable excipient.

As used herein, the terms “enhanced performance of the muscles” or “enhanced muscle performance” mean that the irritability, conductivity, contractility, and/or adaptability of the muscles are better with the use of the nutritional supplements described herein than without. “Enhanced performance of the muscles” or “enhanced muscular performance” includes improvements in muscle strength, endurance, speed, power, or work capacity. During an intensive training period, for example, athletes are expected to experience improved muscle work capacity when using the nutritional supplements disclosed herein. In one embodiment, enhanced muscle performance includes a reduction in pain, stiffness and aches after a bout of exercise or after resistance and endurance training. In another embodiment, enhanced muscle performance results in an increase in lean body mass. In another embodiment, enhanced muscle performance includes a decrease in catabolism, or muscle loss, or muscle atrophy, resulting from exercise. As used herein, muscle atrophy is the partial or complete wasting of a muscle.

In one embodiment, enhanced muscle performance includes a decrease in catabolism, muscle loss or atrophy resulting from exercise.

As used herein, “resistance training” includes “progressive resistance training.” “Progressive resistance training” is resistance exercise in which the overload is progressively increased to facilitate adaptation, for example by increasing the amount of weight lifted and/or the number of sets and/or repetitions, and/or by reducing rest periods between sets. The nutritional supplements are particularly useful to improve the muscle building effects of resistance training.

As used herein, the terms “enhanced recovery of the muscles” or “enhanced muscle recovery” mean that the muscles are restored to normal level of function faster with the use of the nutritional supplements disclosed herein than without. In one embodiment, muscle recovery includes muscle repair, such as the repair of normal muscle damage associated with exercise. Normally the symptoms of delayed onset muscle soreness (DOMS) develop during the first 24 to 48 hours following exercise. After the intake of the nutritional supplements disclosed herein, the subjective symptoms of DOMS are expected to be significantly reduced or even disappear, and also shorter recovery periods between individual exercise sessions (workouts) may be required.

The use of the nutritional supplements disclosed herein additionally enhances muscle power output. As used herein, the term “enhanced power” means that the muscles can generate a higher level of force per unit of time when the combination of nutritional agents disclosed herein are used than without.

As used herein, the term “strenuous exercise” refers to physical activity that is performed with a relatively high level of muscle force and/or exertion. The “state of stress induced by physical exercise, disease or trauma” of the muscle means that the muscle is in a metabolic state wherein the net protein balance is negative due to increased protein catabolism and/or reduced protein synthesis (anabolism). In trained muscle this state of stress leads to symptoms of aching, tender, and swollen muscles with reduced range of motion and rigidity, and prolonged strength loss. In trauma this state of stress often leads to immobilization and atrophy of the muscle. Diseases that induce a state of stress in muscles include all diseases or disorders involving muscle cell damage, loss or atrophy such as catabolic conditions and muscular dystrophy.

In one embodiment, enhancing muscle performance and/or recovery includes treating the symptoms post-exercise muscle soreness such as delayed-onset muscle soreness (DOMS). Individuals with DOMS, for example, experience painful, tender, and swollen muscles with a reduced range of motion of adjacent joints, especially after unaccustomed exercise. In addition to muscle tenderness with palpation, prolonged strength loss, a reduced range of motion and elevated levels of serum creatine kinase are observed. These symptoms develop during the first 24 to 48 hours and disappear within 2 to 7 days. DOMS symptoms are particularly associated with the eccentric exercise, i.e., a type of exercise where an activated muscle is forced to lengthen while producing tension.

In one embodiment, enhancing or preserving muscle mass, muscle strength, physical performance, muscle recovery, or a combination thereof includes treating sarcopenia and/or andropause. Such enhancement generally includes muscle preservation. Sarcopenia refers to the loss of muscle mass and strength associated with normal aging. Thus, the methods described herein are particularly useful to treat aging individuals suffering from sarcopenia, or age-related muscle loss and functionality, such as individuals of age 25 and older. Andropause refers to the decline in testosterone associated with normal aging in men. Loss of muscle performance is associated with andropause. Therefore the nutritional supplements disclosed herein can be used to improve muscle performance in men suffering from the effects of andropause, particularly men of age 40 years and over. While the nutritional supplements are particularly beneficial when administered to individuals undergoing exercise training, benefits are expected in the absence of exercise, particularly in the case of sarcopenia and andropause. Thus, the nutritional supplements can be administered with or without exercise.

In another embodiment, muscle preservation includes preservation during diets and treatment of a catabolic condition. For example, the compositions disclosed herein can be administered to individuals who are consuming a sub-maintenance level of energy, such as with a weight-loss diet or when fasting (e.g., overnight, or in between meals). The compositions can thus be used to preserve lean muscle mass in individuals during weight-loss diets or energy-restricted diets. For example, a method of using the compositions described herein includes inhibiting or reducing muscle mass and strength losses associated with weight-loss or energy-restricted diets, fasting, or other conditions where a sub-maintenance level of energy is being consumed.

In one embodiment, the amount of HICA, a physiologically acceptable ester or amide derivative, or salt thereof in the nutritional supplement is about 0.1 to about 3.0 g, specifically about 0.25 to about 2.0 g, or more specifically about 0.5 to about 1.5 g. The amount of aspartic acid or a physiologically acceptable ester or amide derivative or salt thereof in the nutritional supplement is about 0.25 to about 5.0 g, specifically about 0.5 to about 4.0 g, or more specifically about 1.0 to about 3.0 g. The amount of the pharmaceutically acceptable excipient can vary widely and is about 1% to about 99% of the total weight of the nutritional supplement. The nutritional supplement may be administered one or more times daily to provide a muscle performance or recovery enhancing amount of HICA or a physiologically acceptable ester or amide derivative or salt and aspartic acid or a physiologically acceptable ester or amide derivative or salt thereof.

In one embodiment, a typical effective dosage of HICA is less than or equal to 20 mg/kg body weight/day of HICA. For example a 90-kg (198-lb) individual might consume 1800 mg of HICA daily using this guideline. In comparison, many conventional nutritional supplements might provide 100 to 300 grams of HICA per day. The HICA dosage is 5 to 100 bodyweight mg/kg/day, specifically 10 to 40 mg/kg bodyweight/day, and most specifically 15 to 20 mg/kg bodyweight/day. In another embodiment, a typical effective dosage of aspartic acid or a physiologically acceptable ester or amide derivative or salt thereof is less than or equal to 40 mg/kg bodyweight/day of aspartic acid. The aspartic acid dosage is 10 to 200 mg/kg bodyweight/day, specifically 20 to 100 mg/kg bodyweight/day, and most specifically 35 to 45 mg/kg bodyweight/day. However, the dosage may be higher or lower than indicated here, since the suitable dose depends on the individual, the nature and intensity of his or her training (e.g. prolonged endurance training vs. resistance exercise), his or her diet, age, gender and similar factors.

The nutritional supplements disclosed herein may contain active agents in addition to HICA and aspartic acid. Exemplary additional active agents include vitamins, minerals, amino acids, and other dietary supplements or botanical ingredients. In one embodiment, a composition comprises HICA, aspartic acid, Tribulus terrestris and/or Fenugreek and/or zinc and/or magnesium, and optionally a pharmaceutically acceptable excipient.

For enhancement of muscle mass and/or strength, and/or physical performance and/or recovery from exercise, the nutritional supplement is taken after each training session. However, for periodic or long-term use, the timing of the intake is not critical as long as the levels of HICA and aspartic acid in the blood are sufficient to produce the desired benefits. For athletes, sufficient blood levels of HICA and aspartic acid may be achieved by ingesting the nutritional supplement two to four times per day, for instance. Generally, it is suggested that HICA and aspartic acid be taken immediately after the training period, preferably within 1 to 3 hours afterwards. However, the alleviation of delayed onset muscle soreness (DOMS) symptoms may be achieved by the ingestion of HICA and aspartic acid even after up to 24 hours after the training session.

The nutritional supplement is administered by any suitable route, such as orally, intramuscularly or intravenously. In one embodiment, the supplement is administered orally. A suitable dosage format for oral administration is a solid dosage form, such as a lozenge, tablet, capsule, granule, dissolvable oral strip, microgranule or powder, or a liquid dosage form, such as a solution, ready-to-drink beverage, suspension or injectable solution. One solid dosage format for oral administration is a compressed or coated tablet. Other solid formats for oral administration are granules and powders, which can upon use be dissolved in a suitable liquid such as water, juice, milk, or the like. Alternatively, the nutritional supplement can be in the format of a drink mix, bar, soft gel or the like. For intramuscular or intravenous administration, HICA and DAA are dissolved in a solvent suitable for injection, such as physiological saline. A specific liquid format is a ready-to-drink beverage.

The term “pharmaceutically acceptable” as used herein refers to pharmaceutically active agents or inert ingredients which are suitable for ingestion by animals, including humans, without undue toxicity, incompatibility, instability, irritation, allergic response, and the like, commensurate with a reasonable benefit/risk ratio.

The terms excipient and carrier are used interchangeably herein. Exemplary pharmaceutically acceptable excipients include inert diluents, such as calcium carbonate, sodium carbonate, sodium citrate, lactose, calcium phosphate, sodium phosphate, microcrystalline cellulose, corn starch, potato starch, and cellulose esters such as cellulose acetate, ethyl cellulose; granulating and disintegrating agents, for example, corn starch, or alginic acid, or complex silicates; binding agents, for example starch, polyvinylpyrrolidone, PEG-8000, gelatin or gum acacia, and lubricating agents, for example magnesium stearate, stearic acid, sodium lauryl sulfate, or talc. Nutritional supplements can also include sweetening agents, flavoring agents, coloring agents, preservative agents, stabilizers, buffers, dispersants, thickeners, solubilizing agents, and antioxidants.

The compositions can, if desired, be presented in a pack or dispenser device which can contain one or more units containing the active ingredient(s). The pack can for example comprise metal or plastic foil, such as a blister pack. The pack or dispenser device can be accompanied by instructions for administration.

In one embodiment, the nutritional supplement is administered to a mammal, such a human, a horse, a cat or a dog. In a specific embodiment the mammal is a human, and more specifically a human athlete.

The invention is further illustrated by the following non-limiting examples

### EXAMPLES

A four-arm clinical study will demonstrate the efficacy of the combination of HICA and aspartic acid in the enhancement and recovery of muscle as well as the prevention of muscle catabolism. In this study, two groups of test subjects, men between the ages of 25 and 54, will take the HICA and DAA combination orally, with one group taking the combination with a manner of daily resistance exercise and the other group taking just the combination of ingredients orally without exercise. The third arm of the study will be a control group, taking a placebo without exercise and the fourth group will take a placebo with exercise. This clinical trial will consist of taking the combination or placebo daily for a period of at least 30 days. Subjects in the exercise groups will complete a daily log detailing their exercise, including type of exercise, weight used, repetitions and other standard exercise logs, plus a self-assessment of muscle soreness to determine the rate of delayed onset of muscle fatigue. The non-exercise groups will forego such an assessment. All groups, prior to the study and at the study's conclusion, will be measured for weight, body mass index, lean body mass, and muscle mass, and a pre- and post-study comparison will be made individually and for each group. All groups will also be measured before the study and at the study's conclusion, via blood and urine draws, for testosterone, estrogen and related hormone measures as well as urea nitrogen levels, which will allow for drawing conclusions on the products individual and synergistic effects.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

All ranges disclosed herein are inclusive and combinable. While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

1. A method for enhancing muscle mass, muscle strength, physical performance, recovery from exercise, or a combination thereof in a mammal, comprising
administering to the mammal, before exercise, during exercise, after exercise, or a combination thereof, a nutritional supplement comprising:
α-hydroxy-isocaproic acid, a physiologically acceptable ester or amide derivative of α-hydroxy-isocaproic acid, or a salt of any one of the foregoing;
aspartic acid, a physiologically acceptable ester or amide derivative of aspartic acid, or a salt of any one of the foregoing; and
optionally a pharmaceutically acceptable carrier or excipient.

2. The method of claim 1, wherein administering is after strenuous physical exercise.

3. The method of claim 1, wherein administering reduces pain, stiffness and aches after a bout of exercise, resistance training, or endurance training.

4. The method of claim 3, wherein the resistance training is progressive resistance training.

5. The method of claim 1, wherein administering also improves power performance.

6. The method of claim 1, wherein administering increases lean body mass.

7. The method of claim 1, wherein enhanced muscle performance includes a decrease in catabolism, muscle loss, or atrophy resulting from exercise.

8. The method of claim 1, wherein the mammal is a human athlete.

9. The method of claim 1, wherein the mammal is a human in need of treatment for sarcopenia.

10. The method of claim 1, wherein the mammal is a human male suffering from reduced muscle mass associated with andropause, physical performance associated with andropause, or both.

11. The method of claim 1, wherein the mammal is in need of treatment for exercise-induced muscle soreness.

12. The method of claim 1, wherein -hydroxy-isocaproic acid, physiologically acceptable ester or amide derivative of α-hydroxy-isocaproic acid, or salt is administered in an amount of 5 to 100 mg/kg/day and the aspartic acid, physiologically acceptable ester or amide derivative of aspartic acid, or salt is administered in an amount of 10 to 200 mg/kg/day.

13. The method of claim 1, wherein administering is within 1 to 3 hours of completion of an exercise session.

14. A method of enhancing or preserving muscle mass, muscle strength, physical performance, or a combination thereof in a mammal in need thereof, comprising administering to the mammal a nutritional supplement comprising:
α-hydroxy-isocaproic acid, a physiologically acceptable ester or amide derivative of α-hydroxy-isocaproic acid, or a salt of any one of the foregoing;
aspartic acid, a physiologically acceptable ester or amide derivative of aspartic acid, or a salt of any one of the foregoing; and
optionally a pharmaceutically acceptable excipient,
wherein administering is independent of exercise.

15. The method of claim 14, wherein the individual is in need of treatment for sarcopenia or andropause.

16. The method of claim 14, wherein the individual is consuming a weight-loss or energy-restricted diet, wherein a sub-maintenance level of energy is consumed by the individual.
